(19) United States
(12) Patent Application Publication
(10) Pub. No.: US 2022/0132857 A1
(43) Pub. Date: May 5, 2022

(54) Disinfecting Composition and Related Methods

(71) Applicant: Arash Mirzaei, Toronto (CA)

(72) Inventor: Arash Mirzaei, Toronto (CA)

(21) Appl. No.: 17/518,896

(22) Filed: Nov. 4, 2021

Related U.S. Application Data
(60) Provisional application No. 63/109,512, filed on Nov. 4, 2020.

(51) Int. Cl. A01N 59/00; A01N 65/08; A01N 65/40; A01N 25/22; A01N 25/04; A61K 8/9789; A61K 8/9794; A61Q 17/00; A01P 1/00; A61K 8/22

(52) U.S. Cl. CPC A01N 59/00; A01N 65/08; A01N 65/40; A01N 25/22; A61K 8/22; A61K 8/9789; A61K 8/9794; A61Q 17/005; A01P 1/00; A01N 25/04

(57) ABSTRACT

Described herein is a disinfectant composition comprising a hydrogen peroxide source and a saponin source. An optional stabilizer, such as a metal chelator, and/or a solvent, such as water, may also be included in the compositions described herein. Also described are hand sanitizers and methods for sanitizing surfaces using the composition described herein.

### CROSS-REFERENCE TO RELATED APPLICATION

This application claims priority to U.S. Provisional Application No. 63/109,512; filed Nov. 4, 2020, the content of which is hereby incorporated herein by reference in its entirety.

### FIELD

The present invention relates to disinfecting compositions. More specifically, the present invention is, in aspects, concerned with disinfecting compositions, sprays, sanitizers, and related methods of making and using same.

### BACKGROUND

Prevention of the spread of viral and bacterial infections through contaminated skin and other surfaces has become an essential part of the fight against pandemic outbreaks. During pandemic outbreaks, healthcare facilities and personnel are exposed to infectious viruses and bacteria beyond their capacity and require extensive and frequent disinfection. Gyms, office buildings, restaurants, and grocery stores will also require more frequent handwashing and sanitization.

During the coronavirus disease 2019 (“COVID-19”) pandemic, the strain on healthcare personnel became a critical issue and the need for frequent handwashing shifted from an essential part of the solution to a problem of its own as most existing handwashing products have their own safety shortcomings.

A problem with most of existing handwashing products is the trade-off between disinfection efficacy and skin tolerance. For example, alcohol-based sanitizers are required to contain more than 60% alcohol, and usually up to about 76%, to be considered effective disinfectants, and despite containing other components to control the detrimental effects of the high concentration of alcohol on the skin, the high-concentration level of alcohol can be abrasive on the skin. Frequent skin contacts with these alcoholic sanitizers have highlighted the need for less aggressive yet effective sanitizers.

Another concern with alcohol-based sanitizers arising from the high concentration of alcohol is their flammability. Ethyl and propyl alcohols are highly flammable at high concentrations with the flash point of a typical 63% ethanol-based sanitizers being approximately 70 degrees Fahrenheit or 21 degrees Celsius. This will make the stockpiling of these products a considerable hazard even though maintaining a high inventory level of disinfectants during a pandemic outbreak is vital.

Additionally, the widespread application of alcohol-based disinfectants is raising a reasonable concern about the release of Volatile Organic Compounds (“VOCs”) as their consumption has increased to billions of liters per year.

Attempts have been made in the past to overcome the shortcomings of currently used sanitizers through the incorporation of additives, as in U.S. Pat. No. 6,617,294B2.

Another concern is about the fact that there are very few effective and safe chemical sporicides, particularly in cold soaking instruments sensitive to chemical attack. The most widely used sporicidal solutions are based on aldehydes, short chain alcohols, phenolic compounds and certain peroxgens. Aldehydes (for example, formaldehyde or glutaraldehyde), although highly effective, suffer from serious occupational safety and environmental elimination risks. Of the peroxgens, the peracids are the most widely used in liquid form. Peracetic and performic acids have been marketed for the disinfection of semi-critical and critical instruments; however, their aggressive chemical nature tends to damage surfaces and instruments with prolonged use.

Alcoholic or phenolic compounds that show good efficacy against mycobacterial species are typically not effective in destroying bacterial endospores. Mycobactericidal products based on short chain alcohols typically contain these ingredients at high concentrations (usually more than 20% w/w). This makes the products very flammable and toxic. In addition, they are often characterized by a strong alcoholic odor and are therefore difficult to use in large quantities in small, enclosed spaces by chemically sensitive individuals. Phenolic compounds can be used by themselves or in combination with other active germicides (such as with quaternary ammonium compounds and solvents), to achieve broad spectrum efficacy. These compounds are also very volatile and show strong unpleasant odors.

In another attempt, U.S. Pat. No. 6,927,237 discloses antimicrobial compositions comprising microbicidal solvents, such as benzyl alcohol, diluent solvents, such as water and additional microbicides. Specific examples disclose compositions comprising benzyl alcohol, water, mixtures of peracids and anionic surfactants.

Solutions of hypochlorite and other chlorine-based compounds are effective against both mycobacteria and bacterial endospores; however, they are easily inactivated by the presence of organic matter, are unstable when diluted, have a strong unpleasant smell of chlorine and are very corrosive and therefore, damage most instruments and surfaces.

Aqueous chemical disinfectants are used in applications where, due to work, environmental or toxicological problems, solvent-based solutions cannot be used. While there are a large number of sanitizing and sanitizing solutions available in the market, there is still a need for an aqueous disinfectant of low volatility, low toxicity, non-corrosive, non-irritating and stable that is effective against hydrophilic viruses, acid-resistant bacteria and bacterial endospores.

### SUMMARY

In accordance with an aspect, there is provided a disinfectant composition comprising a hydrogen peroxide source and a saponin source.

In an aspect, the hydrogen peroxide source comprises hydrogen peroxide, sodium percarbonate, potassium percarbonate, sodium perborate, potassium perborate, hydrogen peroxide urea, hydrated forms thereof, or mixtures thereof.

In an aspect, the saponin source comprises a plant extract.

In an aspect, the plant extract comprises an extract of Camellia sinensis, soapwort (Saponaria officinalis), soapberry (Sapindus species), soapbark (Quillaja saponaria), Camellia oleifera, Sapindus mukorossi, sugar beet root saponins, or a mixture thereof.

In an aspect, the saponin is extracted from a nutshell of Sapindus mukorossi.

In an aspect, the saponin is extracted from the defatted seed meal of Camellia oleifera.

In an aspect, the saponin source is Yocca saponin powder.

In an aspect, the composition comprises from about 0.05% to about 8% by weight of the hydrogen peroxide source, such as from about 1% to about 8% by weight for a concentrated composition and from about 0.05% to about 5% by weight for a ready-to-use composition.

In an aspect, the composition comprises from about 0.01% to about 99% by weight of the saponin source, such as from about 1% to about 10% by weight for a concentrated composition and from about 0.05% to about 3% by weight for a ready-to-use composition.

In an aspect, the composition further comprises a solvent.

In an aspect, the solvent comprises an aqueous solvent.

In an aspect, the aqueous solvent comprises distilled water.

In an aspect, the solvent comprises a water-miscible organic solvent.

In an aspect, the water-miscible organic solvent comprises ethanol, propanol, and/or butanol.

In an aspect, the water-miscible organic solvent is present in an amount of from about 0.1% to about 30% by weight of water present in a ready-to-use composition.

In an aspect, the composition further comprises a stabilizer.

In an aspect, the stabilizer comprises a metal chelator.

In an aspect, the metal chelator comprises an amino acid and/or a carboxylic acid.

In an aspect, the amino acid comprises glutamic acid, histidine, glycine and/or lysine and wherein the carboxylic acid comprises acetic acid, gluconic acid, and/or citric acid.

In an aspect, the stabilizer comprises glutamic acid and citric acid.

In an aspect, the stabilizer is present in an amount of from about 0.05% to about 5% by weight such as from about 0.1% to about 5% by weight for a concentrated composition and from about 0.05% to about 1% by weight for a ready-to-use composition.

In an aspect, the composition is free of surfactants other than the saponin source.

In an aspect, the composition is free of synthetic surfactants.

In an aspect, the composition has a pH of from about 3 to about 6, such as from about 4 to about 5.

In an aspect, the composition is a concentrate.

In an aspect, the concentrate is in the form of a powder or tablet.

In an aspect, the composition is ready-to-use.

In an aspect, the composition disinfects a surface more quickly than a matched composition without a saponin source and/or stabilizer.

In an aspect, the composition is effective against bacteria, viruses, fungi, yeast, or combinations thereof.

In an aspect, the composition is effective against pathogens such as MRSA, E. coli, coronaviruses such as SARS-CoV-1, SARS-CoV-2, and MERS-CoV; influenza viruses such as H1N1, H5N1; Acinetobacter baumannii, Escherichia coli, Haemophilus influenzae, Klebsiella pneumoniae, Candida albicans, or combinations thereof.

In accordance with an aspect, there is provided a hand sanitizer comprising the composition described herein.

In accordance with an aspect, there is provided a spray comprising the described herein.

In accordance with an aspect, there is provided a powder or tablet comprising the composition described herein.

In accordance with an aspect, there is provided a method of sanitizing a surface, the method comprising applying the composition described herein.

In an aspect, the surface comprises skin, furniture, medical equipment, fabric, textiles, paper, kitchen surfaces, or combinations thereof.

Other features and advantages of the present invention will become apparent from the following detailed description. It should be understood, however, that the detailed description and the specific examples while indicating embodiments of the invention are given by way of illustration only, since various changes and modifications within the spirit and scope of the invention will become apparent to those skilled in the art from said detailed description.

### DETAILED DESCRIPTION

Described herein is a hydrogen peroxide-based disinfectant either in the form of a concentrate or a ready-to-use disinfectant solution. In typical aspects, the compositions and methods described herein avoid the use of synthetic-based surfactants typically found in accelerated hydrogen peroxide formulations. While the replacement of synthetic-based surfactants with natural-based surfactants is an attempt to address one major public concern about the ever-increasing usage of synthetic surfactants, it is also a leap forward in the efficacy of hydrogen peroxide-based disinfectants, which allows for a broader application of this VOC-free disinfectant.

### Definitions

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” (or vice versa) wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effects described herein. For example, a composition defined using the phrase “consisting essentially of” encompasses any known pharmaceutically acceptable additive, excipient, diluent, carrier, and the like. Typically, a composition consisting essentially of a set of components will comprise less than 5% by weight, typically less than 3% by weight, more typically less than 1% by weight of non-specified components.

It will be understood that any component defined herein as being included may be explicitly excluded by way of proviso or negative limitation, such as any specific compounds or method steps, whether implicitly or explicitly defined herein.

In addition, all ranges given herein include the end of the ranges and also any intermediate range points, whether explicitly stated or not.

Finally, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

### Compositions

Hydrogen peroxide is an oxidizing biocide that releases active oxygen upon contact with viruses and bacteria. Due to the release of this oxidizing active oxygen, hydrogen peroxide is a potent broad-spectrum germicide and a relatively safe one in terms of the long-term effects of the decomposition of hydrogen peroxide-based products on the environment.

While pure and concentrated solutions of hydrogen peroxide are potent and broad-spectrum germicides, they are also dangerous, unstable, and difficult to handle products. For instance, 90% hydrogen peroxide is more dangerous in terms of handling than 90% alcohol. It is one of the most efficient oxidizers in existence and is used for that property in the rocket fuel industry. Hydrogen peroxide solutions stronger than 8% are regulated in terms of shipping and handling and therefore most hydrogen peroxide-based sanitizers are solutions of less than 8% hydrogen peroxide diluted in water.

Dilute solutions of hydrogen peroxide with hydrogen peroxide content of less than 3% suffer from their own shortcomings. Most notably, loss of activity over time and relatively lower disinfection efficacy.

An example of the lower disinfection efficacy of hydrogen peroxide is that a 6 weight percent hydrogen peroxide solution, within 5 minutes of contact time, will only exhibit, approximately a 3 Log (i.e., 1,000-fold) reduction against Staphylococcus Aureus, and less than a 2 Log (i.e., 100-fold) reduction against Pseudomonas Aeruginosa. Likewise, a 6,000 parts per million (“ppm”) (i.e., 0.6 wt. %) hydrogen peroxide solution, within a 5-minute contact time, will only exhibit about a 1 Log (i.e., 10-fold) reduction against Staphylococcus Aureus, and less than a 1 Log reduction against Pseudomonas Aeruginosa.

The disinfection efficacy of hydrogen peroxide can be improved by using more concentrated hydrogen peroxide or a longer contact time. Stronger solutions with hydrogen peroxide content of higher than 3% and lower than 8% remain unstable and at a concentration higher than 7.5 wt. % are considered corrosive to certain metals that are extensively used in the healthcare industry. As a result, special handling procedures are required for such concentrations. Additionally, contact times longer than 5 minutes are generally not acceptable for most disinfectant applications.

Because of these difficulties in increasing disinfection efficacy, efforts have been made on developing activated or accelerated hydrogen peroxide formulations to improve the efficacy and killing rate of hydrogen peroxide without having to resort to high hydrogen peroxide concentrations and contact times greater than 5 minutes. The development of these activated or accelerated hydrogen peroxide formulations is especially important to industries such as the food, healthcare, hospitality, and even household use.

One attempt to enhance the efficacy of hydrogen peroxide disinfecting solutions is found in U.S. Pat. No. 5,523,012 to Winterton et al. Winterton discloses that the addition of anionic sulfosuccinate surfactants to an aqueous, hydrogen peroxide solution improved the killing time against Aspergillus fumigatus by lowering it to 7.1 minutes. However, the improvement in killing time remains far too low for most disinfectant applications.

Another attempt to enhance the efficacy of hydrogen peroxide disinfecting solutions is found in U.S. Pat. No. 5,264,229 to Mannig et al. Mannig discloses a process for reducing the overall bacterial count and increasing the shelf life of the aqueous hydrogen peroxide solution by adding sulfo-based surfactants, such as, alkylaryl sulfonates, sulfates, sulfonates of oils and fatty acid, sulfate of alcohols and sulfosuccinates.

In U.S. Pat. No. 7,658,953, a biocidal hydrogen peroxide composition is enhanced by the addition of a surfactant with the structure: R—0—(CH(Y)—CH2—0)n—CH2—COOH, wherein R is C6 to C12 alkyl, Y is H or CH3, and n is 3-10. The addition of the surfactant was reported to achieve a Log 6 reduction of bacteria in a bacteria suspension within 1-minute following contact with the composition.

Several other patents and patent publications disclose the addition of anionic surfactants based on sulfonic acid to increase the kill rate of an aqueous hydrogen peroxide disinfecting solution: U.S. Pat. No. 7,354,604, U.S. Publication Nos.: 2010/0330196, 2011/0262557, 2011/0129435, 2011/0182958, 2012/0230869, 2012/0177746, 2012/0164237, and European Publication No.: 2338343. The anionic surfactant is chosen from C8 to C16 alkyl aryl sulfonic acids, sulfonated C12 to C22 carboxylic acids, Ce to C22 alkyl diphenyl oxide sulfonic acids, C8 to C22 alkyl sulfonic acids, and Ce to C18 alkyl or alkenyl esters of sulfosuccinic acids. However, the use of anionic sulfonic acid-based surfactants is less desirable in applications where disinfection, and not cleaning, is the primary function to be achieved.

Furthermore, hydrogen peroxide solutions suffer from loss of activity over time. As the physical chemistry of hydrogen peroxide suggests, and is confirmed through practical monitoring, hydrogen peroxide solutions tend to dissociate and release oxygen over time, converting to water and nascent active oxygen. High-strength hydrogen peroxide solutions can go through this dissociation process rapidly and, occasionally, violently. Low-strength hydrogen peroxide solutions dissociate more slowly but, nevertheless, lose their activity over a long storage period. The mechanisms through which this dissociation happens are categorized as metal catalyzed reactions, in which multivalent metals can catalyze hydrogen peroxide decomposition through chemical absorption of oxygen to convert to higher electronic states; and pH, in which in an alkaline solution hydrogen peroxide loses its activity more rapidly. While keeping the hydrogen peroxide solutions at a low pH through acid addition is one way of stabilizing the solution, the effect of low pH on the skin is not desirable, and can be even detrimental in extremely low levels.

Saponins are a large and structurally diverse group of bioactive natural products that are found primarily in plants, most commonly within the dicots. They are compounds constructed of a triterpene or steroid moiety (aglycon or sapogenin) and one or two glycoside moieties (monodesmosides or bidesmosides, respectively). The aglycon carbon skeleton may be saturated or unsaturated and/or contain a heteroatom, such as nitrogen. The glycoside moiety contains sugars, such as galactose, glucose, glucuronic acid, methylpentose, rhamnose and xylose.

It has now been found that saponins act as an effective additive to enhance and accelerate the biocidal functionality of hydrogen peroxide-containing solutions and thereby help alleviate a major disadvantage of hydrogen peroxide as a sanitizer.

As another benefit, saponins add natural-based foaming and detergency to hydrogen peroxide-containing disinfectants thereby eliminating the need for synthetic surfactants.

Thus, described herein is a disinfectant composition comprising a hydrogen peroxide source and a saponin source.

Any source of hydrogen peroxide is contemplated for use herein. Typically, however, the hydrogen peroxide source comprises hydrogen peroxide, sodium percarbonate, potassium percarbonate, sodium perborate, potassium perborate, hydrogen peroxide urea, hydrated forms thereof, or mixtures thereof. In aspects, the hydrogen peroxide source is an aqueous solution containing about 20% to about 50% by weight hydrogen peroxide dissolved in water. In other aspects, the hydrogen peroxide source is a solid formulation of sodium percarbonate.

As will be apparent to those skilled in the art, the amount of hydrogen peroxide used in the composition will vary depending on the application and whether the composition is a concentrate or a ready-to-use solution. The amount of the hydrogen peroxide in the concentrate is typically from about 1.0 to about 8.0 w/w % of the total formulation depending on storage stability, such as from about 1.0, about 1.5, about 2.0, about 2.5, about 3.0, about 3.5, about 4.0, about 4.5, about 5.0, about 5.5, about 6.0, about 6.5, about 7.0, or about 7.5 to about 1.5, about 2.0, about 2.5, about 3.0, about 3.5, about 4.0, about 4.5, about 5.0, about 5.5, about 6.0, about 6.5, about 7.0, about 7.5, or about 8.0 w/w %. Typically, the concentrate contains from about 2 w/w % to about 7 w/w % of the hydrogen peroxide.

In contrast, the amount of hydrogen peroxide in the ready-to-use solution is a biocidal amount that can range from about 0.05 w/w % to about 5.0 w/w % of the total solution depending on application, such as from about 0.05, about 0.1, about 1.5, about 2.0, about 2.5, about 3.0, about 3.5, about 4.0, or about 4.5 to about 0.1, about 1.5, about 2.0, about 2.5, about 3.0, about 3.5, about 4.0, about 4.5, or about 5.0 w/w %. Typically, the biocidal amount ranges from about 0.05 w/w % to about 2.0 w/w %. Typically, the ready-to-use solution contains from 0.1 to 1.5 w/w % of the hydrogen peroxide.

Saponin sources for use herein are diverse and the origin and extraction process of the saponin ingredient is not limiting. Typically, the saponin material is an extract of Camellia sinensis, soapwort (Saponaria officinalist), soapberry (Sapindus species), soapbark (Quillaja saponaria), Camellia oleifera, Sapindus mukorossi, sugar beet root saponins, or a mixture thereof.

The saponin material obtained from a plant source, e.g., Camellia oleifera, and/or Sapindus mukorossi, may be extracted as known in the art or as explained below as a reference only. The extraction process as presented for reference herein typically comprises treating the plant source in a water/alcohol solution under conditions permitting extraction of the saponin material into the solution. The extracted saponin containing material may subsequently be purified by any means known in the art, including filtration, centrifugation, re-crystallization, distillation, adsorption, chromatographic methods, fractionation, etc.

The plant source may or may not be first dried and ground prior to being treated in the water/alcohol solution.

As an example, the saponin material can be extracted from a plant source following a method comprised of:

1. treating the plant source in a 40:60 to 60:40 water: alcohol solution for a period of time and under conditions permitting extraction of the saponin material from said plant source into said solution;

2. optionally, evaporating the saponin-containing solution to obtain a saponin-containing solid material; and

3. optionally, purifying said saponin-containing solid material.

In some aspects, the plant source is Sapindus mukorossi and the saponin material is extracted from the nutshell. In other aspects, the plant source is Camellia oleifera and in some embodiments the saponin material is extracted from the defatted seed meal of Camellia oleifera. In other aspects, the saponin source is Yocca saponin powder.

In typical aspects, the amount of the saponin material of the total weight of the solid materials in the extract composition is in the range of 0.01-99 wt %.

As will be apparent to those skilled in the art, the amount of saponin used in the composition will vary depending on the application and whether the composition is a concentrate or a ready-to-use solution. The amount of the saponin material in the concentrated product is typically from about 1 w/w % to about 10 w/w % of the total formulation, such as from about 1.0, about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, or about 9.5 to about 1.5, about 2.0, about 2.5, about 3.0, about 3.5, about 4.0, about 4.5, about 5.0, about 5.5, about 6.0, about 6.5, about 7.0, about 7.5, about 8.0, about 8.5, about 9.0, about 9.5, or about 10.0 w/w %. Typically, the concentrate contains from 1 w/w % to 5 w/w % of the saponin extract.

The amount of saponin extract in the ready-to-use solution is from about 0.05 w/w % to about 5 w/w % of the total solution, such as from about 0.05, about 0.1, about 1.5, about 2.0, about 2.5, about 3.0, about 3.5, about 4.0, or about 4.5 to about 0.1, about 1.5, about 2.0, about 2.5, about 3.0, about 3.5, about 4.0, about 4.5, or about 5.0 w/w %. Typically, the ready-to-use solution contains from about 0.5 w/w % to about 3 w/w % of the saponin extract.

Typically, the composition further comprises a solvent, particularly in the case of the ready-to-use composition. Typically, the solvent is an aqueous solvent, such as water, typically distilled water, and/or a water-miscible organic solvent, which is typically natural. Water-miscible organic solvents for use herein include, for example: Bio-based alcoholic solvents, such as ethanol, propanol, and butanol, with bio-based ethanol being the typical organic solvent. When the water-miscible organic solvent is used with water, it can be diluted in a range with water from about 0.1% to about 30% by weight of water present in the ready-to-use solution, such as from about 0.1, about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, or about 29 to about 0.5, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29 or about 30%.

In typical aspects, the composition described herein comprises a stabilizer. Typically, the stabilizer comprises a metal chelator that is typically of natural origin. Some amino acids as well as some carboxylic acids have been found to be effective metal chelators in the compositions described herein. Metal chelating amino acids and amino acid peptides generally include, but are not limited to, glutamic acid, histidine, glycine, and lysine. Carboxylic acids with chelation properties might be used alone or in combination with the above-mentioned amino acids for metal chelation. These carboxylic acids include, but are not limited to, acetic acid, gluconic acid, and citric acid.

The concentrated product prepared according to this application may contain from about 0.1% to about 5% of the metal chelator of choice, such as from about 0.1, about 1.5, about 2.0, about 2.5, about 3.0, about 3.5, about 4.0, or about 4.5 to about 1.5, about 2.0, about 2.5, about 3.0, about 3.5, about 4.0, about 4.5, or about 5.0 w/w %. The diluted, ready-to-use product may contain from 0.05 to as much as 1% of the metal chelator, such as from about 0.05, about 0.1, about 0.2, about 0.3, about 0.4, about 0.5, about 0.6, about 0.7, about 0.8, or about 0.9 to about 0.1, about 0.2, about 0.3, about 0.4, about 0.5, about 0.6, about 0.7, about 0.8, about 0.9, or about 1.0 w/w %.

In typical aspects, the composition is free of surfactants other than the saponin source. In additional or alternative aspects, the composition is free of synthetic surfactants.

The composition typically has a pH of from about 3 to about 6, such as from about 4 to about 5, such as from about 3, about 4, or about 5 to about 4, about 5, or about 6.

The composition may be provided as a concentrate or a ready-to-use formulation. Typically, the concentrate is a liquid or powder. The powder may be formulated as a tablet, a capsule, or granules, for example.

The composition typically disinfects a surface more quickly than a matched composition without a saponin source and/or stabilizer and is typically effective against bacteria, viruses, fungi, yeast, or combinations thereof. For example, the composition is in aspects effective against pathogens such as MRSA, E. coli, coronaviruses such as SARS-CoV-1, SARS-CoV-2, and MERS-CoV; influenza viruses such as H1N1, H5N1; Acinetobacter baumannii, Escherichia coli, Haemophilus influenzae, Klebsiella pneumoniae, Candida albicans, or combinations thereof.

Also provided are products comprising the composition described herein. For example, a hand sanitizer comprising the composition is provided, as well as a spray, a powder, or a tablet. In other aspects, the composition is, for example, in the form of a gel, cream, ointment, paste, lotion, or spreadable solid.

Also provided are methods of sanitizing or disinfecting a surface, wherein the methods comprises applying the composition described herein to the surface for a period of time, such as from about 1 second to about 30 minutes, such as from about 1 second, about 10 seconds, about 30 seconds, about 1 minute, about 2 minutes, about 5 minutes, about 10 minutes, about 15 minutes, about 20 minutes, or about 25 minutes to about 10 seconds, about 30 seconds, about 1 minute, about 2 minutes, about 5 minutes, about 10 minutes, about 15 minutes, about 20 minutes, about 25 minutes, or about 30 minutes Typically, the surface comprises skin, furniture, medical equipment, fabric, textiles, paper, kitchen surfaces, or combinations thereof.

The compositions may comprise additional agents and/or excipients as will be understood to a skilled person. For example, buffers such as EDTA may be included, pH adjusting excipients or buffers such as sodium bicarbonate may be included, and/or fragrances such as essential oils may be included in the compositions described herein.

The above disclosure generally describes the present invention. A more complete understanding can be obtained by reference to the following specific Examples. These Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Methods

### Stability of Solutions

A stock solution of 3% hydrogen peroxide was prepared from non-stabilized Fisher brand 30% solution of hydrogen peroxide and divided into six equal aliquots. To one aliquot, the above stabilizer package was added at a concentration of 0.1% and 0.5% and saponin was added to another two at the same concentration. The test containers were sealed and kept at ambient temperature for a period of 60 days, after which the containers were opened and the contents were examined for hydrogen peroxide content (biocidal activity). Two methods were used for the determination of assay hydrogen peroxide, as more particularly described herein.

### Disinfection Efficacy

Bioluminescence is a practical and simple method used to quickly assess the bio-contamination of surfaces. The method has been developed based on the biochemical adenosine triphosphate (“ATP”), which is the energy molecule of all the living organisms, luciferin/luciferase, and oxygen. The reaction results in a light-emitting decay which can be detected and related to the amount of ATP. ATP monitoring is used in food and beverage facilities to confirm that ATP presence is eliminated or minimized by effective sanitation procedures.

The luminometer (in conjunction with ATP swabs) uses bioluminescence to detect residual ATP as an indicator of surface cleanliness. The presence of ATP on a surface indicates incomplete cleaning and the presence of ATP-containing contamination including bacteria.

Because ATP is present in all organic matter, all foods contain ATP at varying levels. Therefore, the surfaces to be evaluated had to be thoroughly cleaned and tested for contamination prior to commencing the test.

Test results are presented as Relative Light Units (“RLU”). RLU is the unit of measurement used in bioluminescence. When a test swab is activated, a bioluminescent reaction occurs, generating light output. Luminometers measure and quantify that light with an RLU output. Because manufacturers use different sensor technologies and algorithms for adding up the photons, RLU measurements will vary from system to system. However, because the ATP bioluminescence reaction is linear, the more ATP present means the more light will be present.

The relationship between the amount of ATP on the sample and the RLU result reading on the luminometer is directly proportional to the amount of ATP collected from the sample. A high RLU reading indicates a large amount of ATP at the test location. This in turn indicates improper cleaning and the presence of contaminants.

Cleaning properly results in less ATP at the location. Less ATP results in less light output during the bioluminescent reaction and consequently, a lower RLU reading.

### Example 1

A granite floor tile was selected and thoroughly disinfected with hot water and a cocktail of disinfectants. It was then tested for ATP until the readings consistently indicated RLU of 0-2 which represented a surface free of ATP within the margins of error.

A sample of seemingly contaminated water was collected from a pond and left stagnant for solid settlement for 3 days.

The granite tile was covered with a paper towel and the water was added to the towel in a dropwise manner until it was completely soaked. After a few hours, soaking was repeated, and the specimen was left to dry overnight. The next morning, the towel was removed, and two equal surface areas were selected and tested for ATP, the results in RLU were as follows:

### Area 1: 1057

### Area 2: 998

Two 50 ml aliquots were taken from a bottle of fresh 3% hydrogen peroxide solution. 0.5 grams of Yocca saponin extract together with 14.5 ml of boiled and cooled distilled water was added to one aliquot such that the active material in each aliquot remained practically at the same level. Two pieces of paper towels were cut to cover each test area and put on top of each area. Area 1 was soaked with 3 ml of hydrogen peroxide with no other additive while area 2 was soaked with 3 ml of saponin containing hydrogen peroxide. After 5 minutes, both papers were removed, and the areas were tested again for ATP with results as follows:

### Area 1: 82

### Area 2: 22

### Example 2

Another sample of water was collected from a different location and was poured on a washed and dried polyester fabric until the fabric was soaked and ambient-dried 2 times over a 24-hour period. Two equal and separate areas were selected and swabbed for ATP with following RLU readings:

### Area 1: 243

### Area 2: 291

Area 1 was soaked in a dropwise manner with 5 ml of the saponin containing hydrogen peroxide solution and area 2 was treated equally with 5 ml of commercial 70% Iso-Propyl Alcohol. After 5 minutes, the two areas were swabbed again for ATP and the related RLU results were record as:

### Area 1: 7

### Area 2: 22

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

1. A disinfectant composition comprising a hydrogen peroxide source and a saponin source.

2. The composition of claim 1, wherein the hydrogen peroxide source comprises hydrogen peroxide, sodium percarbonate, potassium percarbonate, sodium perborate, potassium perborate, hydrogen peroxide urea, hydrated forms thereof, or mixtures thereof.

3. The composition of claim 1, wherein the saponin source comprises a plant extract.

4. The composition of claim 3, wherein the plant extract comprises an extract of Camellia sinensis, soapwort (Saponaria officinalis), soapberry (Sapindus species), soapbark (Quillaja saponaria), Camellia oleifera, Sapindus mukorossi, sugar beet root saponins, or a mixture thereof.

5. The composition of claim 4, wherein the saponin is extracted from a nutshell of Sapindus mukorossi.

6. The composition of claim 4, wherein the saponin is extracted from the defatted seed meal of Camellia oleifera.

7. The composition of claim 3, wherein the saponin source is Yocca saponin powder.

8. The composition of claim 1, wherein the composition comprises from about 0.05% to about 8% by weight of the hydrogen peroxide source, such as from about 1% to about 8% by weight for a concentrated composition and from about 0.05% to about 5% by weight for a ready-to-use composition.

9. The composition of claim 1, wherein the composition comprises from about 0.01% to about 99% by weight of the saponin source, such as from about 1% to about 10% by weight for a concentrated composition and from about 0.05% to about 3% by weight for a ready-to-use composition.

10. The composition of claim 1, further comprising a solvent.

11. The composition of claim 10, wherein the solvent comprises an aqueous solvent.

12. The composition of claim 11, wherein the aqueous solvent comprises distilled water.

13. The composition of claim 10, wherein the solvent comprises a water-miscible organic solvent.

14. The composition of claim 13, wherein the water-miscible organic solvent comprises ethanol, propanol, and/or butanol.

15. The composition of claim 13, wherein the water-miscible organic solvent is present in an amount of from about 0.1% to about 30% by weight of water present in a ready-to-use composition.

16. The composition of claim 1, further comprising a stabilizer.

17. The composition of claim 16, wherein the stabilizer comprises a metal chelator.

18. The composition of claim 17, wherein the metal chelator comprises an amino acid and/or a carboxylic acid.

19. The composition of claim 18, wherein the amino acid comprises glutamic acid, histidine, glycine and/or lysine and wherein the carboxylic acid comprises acetic acid, gluconic acid, and/or citric acid.

20. The composition of claim 16, wherein the stabilizer comprises glutamic acid and citric acid.

21. The composition of claim 16, wherein the stabilizer is present in an amount of from about 0.05% to about 5% by weight such as from about 0.1% to about 5% by weight for a concentrated composition and from about 0.05% to about 1% by weight for a ready-to-use composition.

22. The composition of claim 1, wherein the composition is free of surfactants other than the saponin source.

23. The composition of claim 1, wherein the composition has a pH of from about 3 to about 6, such as from about 4 to about 5.

24. The composition of claim 1, wherein the composition is a concentrate.

25. The composition of claim 1, wherein the composition is ready-to-use.

26. The composition of claim 1, wherein the composition disinfects a surface more quickly than a matched composition without a saponin source and/or stabilizer.

27. A hand sanitizer comprising the composition of claim 1.

28. A method of sanitizing a surface, the method comprising applying the composition of claim 1 to the surface.

29. The method of claim 28, wherein the surface comprises skin, furniture, medical equipment, fabric, textiles, paper, kitchen surfaces, or combinations thereof.
